(19) United States
(12) Patent Application Publication
(10) Pub. No.: US 2021/0015966 A1
(43) Pub. Date: Jan. 21, 2021

(54) Antibacterial Cellulose Hydrogels and Preparation Method therefor

(71) Applicant: Tianjin University of Science and Technology, Tianjin (CN)

(72) Inventors: Cheng ZHONG, Tianjin (CN); Xiangjun Zhao, Tianjin (CN); Yanwen Zhang, Tianjin (CN); Fazli WAHID, Tianjin (CN); Yanyan Xie, Tianjin (CN); Shiru Jia, Tianjin (CN)

(21) Appl. No.: 17/035,705

(22) Filed: Sep. 29, 2020

(30) Foreign Application Priority Data
Sep. 29, 2019 (CN) 201910933619.0

(51) Int. Cl. A61L 26/00; A61L 15/42; A61L 15/28; A61L 15/20

(52) U.S. Cl. CPC A61L 26/0066; A61L 15/425; A61L 26/008; A61L 15/20; A61L 26/0023; A61L 15/28

(57) ABSTRACT

The disclosure provides a preparation method for a novel biocompatible antibacterial bacterial cellulose Fmoc-F hydrogel, belonging to the technical field of biomedical gels. The preparation method is characterized by comprising the following steps of (1) preparing a bacterial cellulose (BC) homogenate, (2) mixing the bacterial cellulose with an Fmoc-F solution, and preparing the bacterial cellulose/Fmoc-F antibacterial hydrogel by means of in-situ chemical crosslinking. The preparation method has the advantages of rapid gelling reaction, simple reaction system, high gel plasticity and the like. Because of the bacterial cellulose, the antibacterial hydrogel prepared in the disclosure has high mechanical strength. Meanwhile, compared with traditional micro-molecular antibacterial materials, the bacterial cellulose/Fmoc-F antibacterial hydrogels prepared in the disclosure have broad-spectrum antibacterial effects and good biocompatibility.

### CROSS REFERENCE TO RELATED APPLICATION

This application claims priority of Chinese Application No. 201910933619.0 filed on Sep. 29, 2019 and entitled “antibacterial cellulose hydrogels and preparation method therefor”.

### TECHNICAL FIELD

The disclosure relates to the technical field of biomedical gels, in particular to a preparation method for a biocompatible antibacterial bacterial cellulose/Fmoc-L-phenylalanine hydrogel.

### BACKGROUND ART

Nowadays, human beings have made great progress in the field of medical treatment, but infectious diseases caused by pathogens such as viruses, bacteria and fungi are still a major health threat and can translate into a wide range of socio-economic problems. With the development of social economy, people's pursuit of quality of life and health is constantly improving, and antibacterial products are believed to be one of the novel health products with a broad development prospect. Traditional antibacterial materials are endowed with certain antibacterial property by introducing antibacterial agents (such as antibiotics, quaternary ammonium salts and bactericides) on the surface or the body of the materials through chemical or physical methods. However, due to the fact that the micro-molecular antibacterial agents like the antibiotics are usually specific and prone to resulting in bacterial resistance, it is very urgent to develop novel antibacterial materials with broad-spectrum antibacterial property.

Hydrogels are novel high molecular functional materials with three-dimensional (3D) network structure formed by physically or chemically crosslinking with hydrophilic polymers and can retain a large amount of water inside the polymer network. Due to its excellent biocompatibility, the hydrogels are widely applied to various biomedical material fields such as drug delivery, tissue engineering and wound healing. Antibacterial hydrogels not only have the characteristics of various polymers and good compatibility, but also can effectively resist bacterial infection, inhibit bacterial resistance and promote wound healing. Therefore, the antibacterial hydrogels have recently been applied to the field of medical treatment as a novel wound dressing. As a biocompatible and eco-friendly renewable resource, biopolymer attracts people's attention in the production of the hydrogels. Bacterial cellulose (BC) is a natural biopolymer produced by bacteria and has the unique features such as bioactivity, biodegradability, biocompatibility, toxicity-free, anaphylaxis-free, high mechanical toughness, excellent water retentivity, high purity, high porosity and high crystallinity, which makes it become an important raw material for the biomedical hydrogels. Bacterial cellulose hydrogels can effectively relieve pain, have good adhesion, can effectively prevent bacterial invasion and infection, are beneficial to the growth of skin tissues and promote wound healing rapidly.

Fmoc-L-phenylalanine (Fmoc-F) is phenylalanine protected by Fmoc and is mainly applied to synthesizing polypeptides. Different amino acid protecting groups have great influence on the efficiency and yield of polypeptide synthesis.

Fmoc-phenylalanine can be used for preparing a phenylalanine bacteriostatic agent to inhibit bacterial growth. The hydrogels obtained by dissolving the Fmoc-L-phenylalanine and the bacterial cellulose in PB solution (PH=7.4) and then heating at 80° C. for 30 min, integrates the advantages of Fmoc-L-phenylalanine and bacterial cellulose and has good biocompatibility, mechanical property and bacteriostatic property, accordingly can be widely applied to the field of biomedical materials.

### SUMMARY OF THE INVENTION

The disclosure aims at providing a novel biocompatible antibacterial bacterial cellulose/Fmoc-L-phenylalanine hydrogel and a preparation method therefor. The hydrogels have good and lasting antibacterial activity.

The technical principles of the present disclosure are as follows: bacterial cellulose biopolymers with good biocompatibility and Fmoc-L-phenylalanine are used as main materials to prepare the bacterial cellulose/Fmoc-L-phenylalanine antibacterial hydrogels by means of in-situ chemical crosslinking.

In order to achieve the described purpose and solve the described technical problem, the present disclosure adopts the following technical solutions:

(1) preparing a bacterial cellulose homogenate: crushing bacterial cellulose membranes with tissue homogenizer by means of mechanical homogenization so as to obtain the bacterial cellulose homogenate, centrifuging the obtained homogenate at 10000 rpm/min for 10 min to remove excessive water and the bacterial cellulose homogenate with a concentration of 10 mg/ml (dry weight) is obtained;

(2) preparing a bacterial cellulose/Fmoc-L-phenylalanine hydrogel: at room temperature, weighing 120 mg of Fmoc-F precisely by analytic balance into 50 ml centrifuge tube, adding 4 ml of PB solution into each centrifuge tube for dissolution, adding 4 ml, 8 ml, 12 ml and 16 ml of the BC homogenate into the centrifuge tubes respectively, and then making up to 20 ml with the PB solution; shaking spirally for 1 min, then heating the mixture at 80° C. for 30 min in a water bath kettle, and then placing the mixture at room temperature for 6 h to obtain the BC-Fomc-F hydrogel, wherein the concentration of the Fmoc-F is 6 mg/ml, and the concentrations of the BC are respectively 2 mg/ml, 4 mg/ml, 6 mg/ml and 8 mg/ml.

Further, the concentration of the bacterial cellulose suspension is 10 mg/ml.

Further, the average length of the fiber of the bacterial cellulose suspension is 10-100 microns.

The present disclosure has the advantages that:

(1) The antibacterial BC-Fomc-F hydrogels prepared in the present disclosure have good bacteriostatic effects on Staphylococcus aureus and Bacillus subtilis.

(2) Compared with traditional micro-molecular antibacterial material, the present disclosure provides a preparation method for a broader-spectrum antibacterial composite material.

(3) The antibacterial hydrogels prepared in the present disclosure are non-poisonous and has good biocompatibility, biodegradability and high mechanical strength.

### BRIEF DESCRIPTION OF DRAWINGS

FIG. 1 shows an effect graph of the antibacterial BC-Fomc-F hydrogels prepared in the present disclosure.

FIG. 2 shows an experimental effect graph of the antibacterial BC-Fomc-F hydrogels on the inhibition zones of Bacillus subtilis and Staphylococcus aureus. (BC-0, BC-2, BC-4, BC-6 and BC-8 represent that the final concentrations of the BC are 0 mg/ml, 2 mg/ml, 4 mg/ml, 6 mg/ml and 8 mg/ml respectively.)

FIG. 3 shows a change-to-frequency graph of storage modulus (G′) and loss modulus (G″) of the antibacterial BC-Fomc-F hydrogels of the disclosure measured by rheometer. (BC-0, BC-2, BC-4, BC-6 and BC-8 represent the change-to-frequency graph of storage modulus (G′) and loss modulus (G″).)

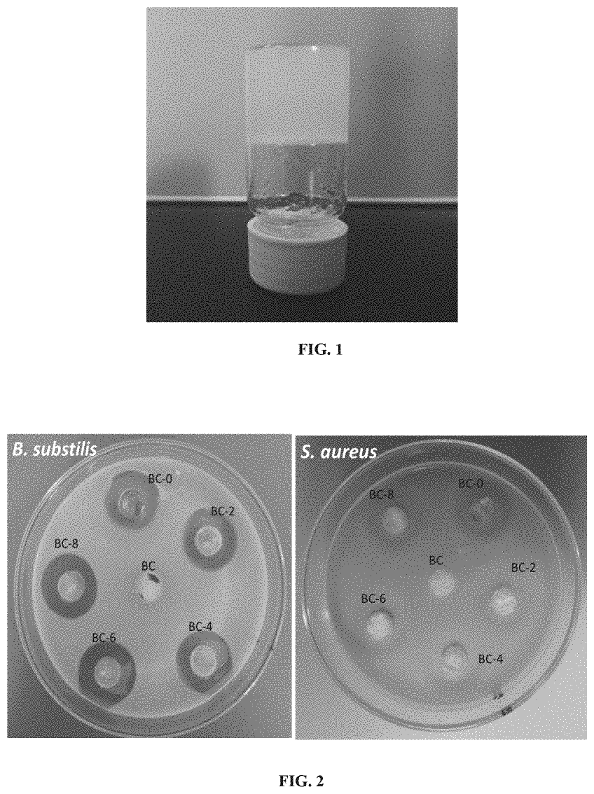

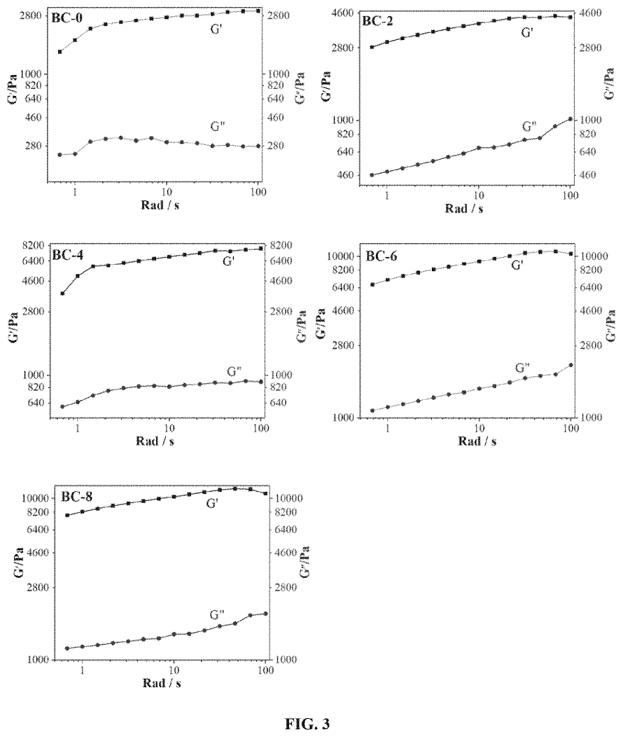

### DETAILED DESCRIPTION

In order to better understand the present disclosure, the following embodiments are provided to further illustrate the present disclosure but not to limit the present disclosure. Based on the embodiments, any other embodiment made by those skilled in the art without making any creative work shall fall within the scope of protection of the present disclosure.

A preparation method for an antibacterial BC-Fomc-F hydrogel, comprising the following steps:

(1) preparing a bacterial cellulose homogenate: crushing bacterial cellulose membranes by means of mechanical homogenization so as to obtain the bacterial cellulose homogenate, centrifuging the obtained homogenate for 10 min to remove excessive water and the bacterial cellulose homogenate with a concentration of 10 mg/ml (dry weight) is obtained;

(2) preparing the bacterial cellulose/Fmoc-L-phenylalanine (BC-Fomc-F) hydrogel: at room temperature, weighing 120 mg of Fmoc-F precisely by analytic balance into 50 ml centrifuge tube, adding 4 ml of PB solution into each centrifuge tube for dissolution, adding 4 ml, 8 ml, 12 ml and 16 ml of the BC homogenate into the centrifuge tubes respectively, and then making up to 20 ml with the PB solution; shaking spirally for 1 min, then heating the mixture at 80° C. for 30 min in a water bath kettle, and then placing the mixture at room temperature for 6 h to obtain the BC-Fomc-F hydrogel, wherein the concentration of the obtained Fmoc-F is 6 mg/ml, and the concentrations of the BC are respectively 2 mg/ml, 4 mg/ml, 6 mg/ml and 8 mg/ml.

### Embodiment 1

A tissue homogenizer is used to crush bacterial cellulose membranes by means of mechanical homogenization so as to obtain the bacterial cellulose homogenate, then the obtained homogenate is centrifuged at 10000 rpm/min for 10 min to remove excessive water, and the bacterial cellulose homogenate with a concentration of 10 mg/ml (dry weight) is obtained.

At room temperature, 120 mg of Fmoc-F is precisely weighed out by an analytic balance into 50 ml centrifuge tube, 4 ml of PB solution is added into the centrifuge tube for dissolution, 4 ml of the BC homogenate is added into the centrifuge tube, and then the centrifuge tube is made up to 20 ml with the PB solution; the solution is shaked spirally for 1 min, then heated at 80° C. for 30 min in a water bath kettle and then placed at room temperature for 6 h to obtain the BC-Fomc-F hydrogel, wherein the concentration of the obtained Fmoc-F is 6 mg/ml, and the concentration of the BC is 2 mg/ml.

### Embodiment 2

A tissue homogenizer is used to crush bacterial cellulose membranes by means of a mechanical homogenization so as to obtain the bacterial cellulose homogenate, then the obtained homogenate is centrifuged at 10000 rpm/min for 10 min to remove excessive water, and the bacterial cellulose homogenate with a concentration of 10 mg/ml (dry weight) is obtained.

At room temperature, 120 mg of Fmoc-F is precisely weighed out using an analytic balance into 50 ml centrifuge tube, 4 ml of PB solution is added into the centrifuge tube for dissolution, 8 ml of the BC homogenate is added into the centrifuge tube, and then the centrifuge tube is made up to 20 ml with the PB solution; the solution is shaked spirally for 1 min, then heated at 80° C. for 30 min in a water bath kettle and then placed at room temperature for 6 h to obtain the BC-Fomc-F hydrogel, wherein the concentration of the obtained Fmoc-F is 6 mg/ml, and the concentration of the BC is 4 mg/ml.

### Embodiment 3

A tissue homogenizer is used to crush bacterial cellulose membranes by means of mechanical homogenization so as to obtain the bacterial cellulose homogenate, then the obtained homogenate is centrifuged at 10000 rpm/min for 10 min to remove excessive water, and the bacterial cellulose homogenate with a concentration of 10 mg/ml (dry weight) is obtained.

At room temperature, 120 mg of Fmoc-F is precisely weighed out using an analytic balance into 50 ml centrifuge tube, 4 ml of PB solution is added into the centrifuge tube for dissolution, 12 ml of the BC homogenate is added into the centrifuge tubes, and then the centrifuge tube is made up to 20 ml with the PB solution; the solution is shaked spirally for 1 min, then heated at 80° C. for 30 min in a water bath kettle and then placed at room temperature for 6 h to obtain the BC-Fomc-F hydrogel, wherein the concentration of the obtained Fmoc-F is 6 mg/ml, and the concentration of the BC is 6 mg/ml.

### Embodiment 4

A tissue homogenizer is used to crush bacterial cellulose membranes by means of mechanical homogenization so as to obtain the bacterial cellulose homogenate, then the obtained homogenate is centrifuged at 10000 rpm/min for 10 min to remove excessive water, and the bacterial cellulose homogenate with a concentration of 10 mg/ml (dry weight) is obtained.

At room temperature, 120 mg of Fmoc-F is precisely weighed out using an analytic balance into 50 ml centrifuge tube, 4 ml of PB solution is added into the centrifuge tube for dissolution, 16 ml of the BC homogenate is added into the centrifuge tube, and then the centrifuge tube is made up to 20 ml with the PB solution; the solution is shaked spirally for 1 min, then heated at 80° C. for 30 min in a water bath kettle and then placed at room temperature for 6 h to obtain the BC-Fomc-F hydrogel, wherein the concentration of the obtained Fmoc-F is 8 mg/ml, and the concentration of the BC is 6 mg/ml.

We claim:

1. A preparation method for an antibacterial BC-Fmoc-F hydrogel, comprising the following steps:
(1) preparing a bacterial cellulose homogenate: crushing bacterial cellulose membranes with tissue homogenizer by means of mechanical homogenization so as to obtain the bacterial cellulose homogenate, centrifuging the obtained homogenate at 10000 rpm/min for 10 min to remove excessive water and the bacterial cellulose homogenate with a concentration of 10 mg/ml (dry weight) is obtained;
(2) preparing a bacterial cellulose/Fmoc-L-phenylalanine hydrogel: at room temperature, weighing 120 mg of Fmoc-F precisely by analytic balance into 50 ml centrifuge tube, adding 4 ml of PB solution into each centrifuge tube for dissolution, adding 4 ml, 8 ml, 12 ml and 16 ml of the BC homogenate into the centrifuge tubes respectively, and then making up to 20 ml with the PB solution; shaking spirally for 1 min, then heating the mixture at 80° C. for 30 min in a water bath kettle, and then placing the mixture at room temperature for 6 h to obtain the BC-Fomc-F hydrogel, wherein the concentration of the Fmoc-F is 6 mg/ml, and the concentrations of the BC are respectively 2 mg/ml, 4 mg/ml, 6 mg/ml and 8 mg/ml.

2. The preparation method for the BC-Fmoc-F hydrogel according to claim 1, wherein the concentration of the bacterial cellulose suspension is 10 mg/ml.

3. The preparation method for the BC-Fmoc-F hydrogel according to claim 1, wherein the average length of the fiber of the bacterial cellulose suspension is 10-100 microns.

4. The preparation method for the BC-Fmoc-F hydrogel according to claim 1, wherein the concentrations of the BC are respectively 2 mg/ml, 4 mg/ml, 6 mg/ml and 8 mg/ml.
